(19) United States
(12) Patent Application Publication
(10) Pub. No.: US 2020/0270656 A1
(43) Pub. Date: Aug. 27, 2020

(54) HIGHLY EFFICIENT ENZYMATIC PROCESS TO PRODUCE (R)-3-QUINUCLIDINOL

(71) Applicant: UNICHEM LABORATORIES LTD, Mumbai (IN)

(72) Inventors: Conchita D'SOUZA, Reis Magos (IN); Maitreyi ASHOK, Caranzalem (IN); Minal P. UDIPI, Solapur (IN); Arijit DAS, Porvorim (IN); Mamata KATDARE, Pune (IN); Sudeep KUMAR, Bopal (IN); Dhananjay SATHE, Thane (IN)

(21) Appl. No.: 16/646,317

(22) PCT Filed: Sep. 5, 2018
(86) PCT No.: PCT/IB2018/056767

§ 371 Date: Mar. 11, 2020

(30) Foreign Application Priority Data
Sep. 12, 2017 (IN) 201721032202

(51) Int. Cl. C12P 17/12

(52) U.S. Cl. CPC C12P 17/12

(57) ABSTRACT

The present invention relates to enzymatic reduction of 3-quinuclidinone to (R)-3-quinuclidinol (Scheme I), by reacting 3-quinuclidinone with a variant of ketoreductase enzyme derived from Rhodotorula rubra. The invention also relates to enzymatically produced (R)-3-quinuclidinol wherein the substrate loading capacity of the enzyme is not less than 100 g/L.

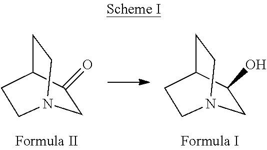 [SMILES: O=C1CN2CCC1CC2 ; O[C@H]1CN2CCC1CC2]

### TECHNICAL FIELD OF THE INVENTION

The present invention relates to a highly efficient enzymatic process to produce (R)-3-quinuclidinol using a recombinant ketoreductase (KRED) expressed in soluble form in Escherichia coli (E. coli).

### BACKGROUND OF THE INVENTION

(R)-3-quinuclidinol of Formula I is an important building block for the production of anti-muscarinic drugs such as Solifenacin succinate, Talsaclidine fumarate, CevimelineHCl.

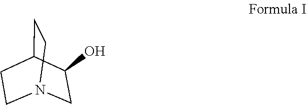

Chemically (R)-3-quinuclidinol is prepared by either reduction of 3-quinuclidinone of Formula II, followed by separation of (R)-3-quinuclidinol or by asymmetric reduction of 3-quinuclidinone or its salt (Scheme I).

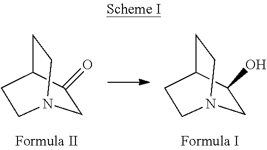 [SMILES: O=C1CN2CCC1CC2 ; O[C@H]1CN2CCC1CC2]

The two step process of reduction of 3-quinuclidinone of Formula II, followed by separation of (R)-3-quinuclidinol is tedious, time consuming, lacks reproducibility while separating the particular enantiomer and is low yielding. The single step conversion using asymmetric chemical catalyst is costly and less reproducible.

There are a few reports on the use of bio-catalysts for conversion of 3-quinuclidinone to (R)-3-quinuclidinol using a co-factor dependent ketoreductase (KRED) enzyme along with glucose dehydrogenase (GDH) enzyme, which is used to regenerate the co-factor. Due to substrate-enzyme specificity, bio-catalytic reaction is reproducible, high yielding without any trace of unwanted enantiomer.

Uzura etal (Appl. Microbiol Biotech (2009) 83:617-626) discloses a method for the production of (R)-3-quinuclidinol with 99% e.e. (enantiomeric excess) The process describes, E. coli strain co-expressing the NAD/NADP (Nicotinamide Adenine Dinucleotide/Nicotinamide Adenine Dinucleotide Phosphate) dependent ketoreductase and a co-factor regenerating enzyme, glucose dehydrogenase (GDH) cloned in two different plasmids, to produce optically active (R)-3-quinuclidinol. The ketoreductase gene was isolated from Rhodotorula rubra JCM3782 and the source of GDH gene was Bacillus megaterium IAM1030. The reaction time is 21 h. Furthermore, the process describes co-expression of the enzymes in the host E. coli BL21 (DE3) (pEQG), E. coli BL21 (DE3) (pEGQ), E. coli W3110 (pTrcQG) and E. coli JM109 (pWKQ, pAG). The article mentions that out of all the above host systems, the activity of E. coli BL21 (DE3) (pEQG) and E. coli BL21 (DE3) (pEGQ) is least and the yield of the product is less. The stereoselective bioconversion in the case of host E. coli W3110 (pWKLQ, pAG) from 3-quinuclidinone (618 millimolar, mM) to (R)-3-quinuclidinol is 98.6% (molar yield) and 99.9% e.e. Under this condition the reaction is slow (21 h). Also the quantity of substrate loading is less (618 mM, ˜63 mg/ml). Thus the process would take longer time and produce lesser amount of product thus making it tedious, inefficient and commercially uneconomical.

Isotani et al [Int. J. Mol Sci. (2012) 13, 13542-13553] describes a reduction system for the production of (R)-3-quinuclidinol using recombinant E. coli cells possessing genes of 3-quinuclidinone reductase from Microbacterium luteolum and the alcohol dehydrogenase of Leifsonia sp. The process was carried out at 150 mg/ml and exhibited high enantioselectivity. The process describes the use of immobilized enzymes for conversion of 3-quinuclidinone to (R)-3-quinuclidinol and organic solvents in the reaction mixture. The process becomes lengthier due to additional cumbersome steps of separation of product from the organic solvents. Use of immobilized enzymes increases the cost of the process.

EP2796548 A1 discloses a process for the preparation of a polynucleotide sequence encoding a chimeric fusion protein comprising a GDH and NADP dependent quinuclidinone reductase amino acid sequence and expressing the said proteins in the host E. coli cells. The process describes the use of whole cell and crude extract containing the chimeric enzymes for the conversion in the presence of phosphate buffer at a suitable pH, temperature and reaction time. The process uses 2-16 g/L of substrate loading and requires 48-96 h for conversion at 30° C. to achieve more than 95% conversion and 99% e.e.

There are several other methods to prepare (R)-3-quinuclidinol from 3-quinuclidinone using ketoreductase derived from microorganism other than Rhodotorula rubra (Rr). In US20140147896 the enzyme is derived from Saccharomyces cerevisiae. EP2423320 uses polypeptide derived from the bacteria Burkholderiasps or a recombinant organism capable of producing the polypeptide. U.S. Pat. No. 5,888,804 discloses a process for the production of optically active (R)-3-quinuclidinol from quinuclidinone using enzyme derived from microorganisms from the genus Nakazawaea. Candida and Proteus.

All the processes mentioned herein are difficult to carry out on an industrial scale. The processes are cumbersome due to lengthy reaction times, low substrate loading, low yield and are expensive. All the processes use pure form of ketoreductase enzyme. Further purification of the enzymes is tedious resulting in compromising yield, longer production time and hence expensive.

Thus, the present invention uses ketoreductase which results in improved yields, reduced process time and hence is cost efficient and commercially viable. The present inventors have thus addressed the long felt unmet need of having an industrially viable and commercially feasible process to prepare (R)-3-Quinuclidinol.

### OBJECT OF THE INVENTION

The main object of the invention is to devise a reproducible, economical, industrially feasible and efficient enzymatic process to produce (R)-3-quinuclidinol.

Another object of the invention is to reduce reaction time of enzymatic conversion of 3-quinuclidinone to (R)-3-quinuclidinol.

Yet another object of the invention is to increase the substrate loading in the reaction having ketorcductasc enzyme for the enzymatic conversion of 3-quinuclidinone to (R)-3-quinuclidinol.

### SUMMARY OF THE INVENTION

The present invention relates to enzymatic reduction of 3-quinuclidinone to (R)-3-quinuclidinol by reacting 3-quinuclidinone with a variant of ketoreductase enzyme derived from Rhodotorula rubra in the presence of a suitable co-factor regenerating system, wherein the variant is in the cell lysate.

The invention further relates to enzymatically produce (R)-3-quinuclidinol of not less than about 99% purity and greater than about 99.5% e.e, wherein the substrate loading capacity of the enzyme is not less than about 100 g/L.

### DETAILED DESCRIPTION OF THE INVENTION

In the present invention the process is said to be highly efficient due to increase in substrate loading capacity of the enzyme which leads to higher yield and reduced time of bioconversion.

The terms ‘ketoreductase enzyme’ ‘3-quinuclidinone reductase’, ‘variant’, are all used interchangeably and all refer to an enzyme derived from the amino acid sequence from Rhodotorula rubra and is used in the enzymatic reduction of 3-quinuclidinone to (R)-3-quinuclidinol.

The substrate used in the present invention is 3-quinuclidinone or salt thereof.

v/v or V/V mean volume/volume; w/v or W/V mean weight/volume and why or W/W mean weight/weight.

Time in hour or hours in indicated as h or hr or hrs. The phrase e.e. is short form of enantiomeric excess and indicates the percentage of one enantiomer compared to the other.

The terms glucose dehydrogenase and GDH are interchangeably used and refers to enzyme derived from the amino acid sequence from Bacillus megaterium (Bin), wherein GDH has the ability to regenerate co-factors like NAD or NADP using glucose as the substrate.

The main embodiment of the present invention relates to the enzymatic reduction of 3-quinuclidinone to (R)-3-quinuclidinol, by reacting 3-quinuclidinone with a variant of ketoreductase enzyme derived from Rhodotorula rubra in the presence of a suitable co-factor regenerating system consisting of a variant of glucose dehydrogenase derived from Bacillus megaterium and co-factors NAD or NADP, wherein both the variants are in the cell lysate.

According to the embodiment the co-factor regenerating system comprises a variant of glucose dehydrogenase and a co-factor NAD or NADP.

According to the aspect of this embodiment the enzymatic reduction of 3-quinuclidinone to (R)-3-quinuclidinol was carried out using the process comprising steps of:
a. reacting 3-quinuclidinone with the cell lysate containing 3-quinuclidinone reductase, in the presence of cell lysate containing glucose dehydrogenase, to give (R)-3-quinuclidinol;
b. extracting and purifying (R)-3-quinuclidinol obtained in step ‘a’.

According to this embodiment of invention 3-quinuclidinone is reacted with cell lysate containing 3-quinuclidinone reductase in the presence of cell lysate containing glucose dehydrogenase to obtain (R)-3-quinuclidinol, wherein the reaction mixture comprises:
a) 3-quinuclidinone as substrate
b) cofactor NAD or NADP;
c) glucose, 1 to 2 times of the substrate concentration;
d) cell lysate containing the enzyme 3-quinuclidinone reductase:
e) cell lysate containing the enzyme glucose dehydrogenase;
f) potassium phosphate buffer medium

Another aspect of this embodiment is a process for extracting and purifying (R)-3-quinuclidinol. The process comprises the steps of:
a) basifying/acidifying the reaction mixture, to obtain basified/acidified reaction mixture;
b) adding acetone to the basified/acidified reaction mixture in step ‘a’, filtering the solvent mixture and removing acetone by evaporation to obtain aqueous solution of product;
c) alternatively, adding celite to the basified/acidified reaction mixture in step ‘a’, stirring at about 20° C.-30° C. for about 20 min to 2 h and filtering to obtain aqueous solution of product;
d) extracting the product from the aqueous solution obtained in step ‘13’ or ‘c’ using n-butanol and concentrating the extract to dryness to obtain the product;
e) solubilizing the product obtained in step in hot toluene.
f) filtering the solution obtained in step ‘c’, gradually cooling the filtrate under constant stirring to room temperature to obtain pure crystals of (R)-3-quinuclidinol and recovering the crystals by filtration.

Yet another embodiment of the invention is to enzymatically produce (R)-3-quinuclidinol of not less than about 99% purity and more than about 99.5% e.e., wherein the substrate loading capacity of the enzyme is not less than about 100 g/L.

According to this embodiment the substrate loading capacity of the enzyme is at least 125 g/L, preferably at least 150 g/L and most preferably at least 175 g/L.

Accordingly, (R)-3-quinuclidinol was produced by reduction of 3-quinuclidinone using a variant of kctoreductase enzyme derived from Rhodotorula rubra in the presence of a suitable co-factor regenerating system.

The co-factor regenerating system comprises variant of glucose dehydrogenase and a co-factor NAD or NADP.

The enzymatic reduction of 3-quinuclidinone to (R)-3-quinuclidinol was carried out using the process comprising steps of:
a) reacting 3-quinuclidinone with at least 4 units of 3-quinuclidinone reductase enzyme per ml of cell lysate, in the presence of cell lysate containing not less than 250 units of glucose dehydrogenase enzyme per milliliter of lysate, wherein 3-quinuclidinone loaded in the reaction wasnot less than 100 g/L;
b) extracting and purifying (R)-3-quinuclidinol obtained in step ‘a’;

According to this embodiment 3-quinuclidinone is reducedusing at least 4 units of 3-quinuclidinone reductase per milliliter of cell lysate in the presence of cell lysate containing not less than 250 units of glucose dehydrogenase to obtain the product (R)-3-quinuclidinol, wherein the reaction mixture comprises:
a) 3-quinuclidinone as substrate, not less than about 100 g/L of reaction mixture;
b) co-factor NAD or NADP;
c) glucose, 1 to 2 times of the substrate concentration;
d) cell lysate containing the enzyme 3-quinuclidinone reductase;
e) cell lysate containing the enzyme glucose dehydrogenase;
f) potassium phosphate buffer.

Another aspect this embodiment is a process for extracting and purifying (R)-3-quinuclidinol. The process comprises the steps of:
a. basifying/acidifying the reaction mixture, to obtain basified/acidified reaction mixture;
b. adding acetone to the basified/acidified reaction mixture in step ‘a’, filtering the solvent mixture and removing acetone by evaporation to obtain aqueous solution of product;
c. alternatively, adding celite to the basified/acidified reaction mixture in step ‘a’, stirring at about 20° C.-30° C. for about 20 min to 2 h and filtering to obtain aqueous solution of product:
d. extracting the product from the aqueous solution obtained in step ‘b’ or in step ‘c’ using n-butanol and concentrating the extract to dryness to obtain the product;
e. solubilizing the product obtained in step ‘d’ in hot toluene at about 80° C.-105° C. to obtain solution;
f. filtering the solution obtained in step ‘e’, gradually cooling the filtrate to room temperature to obtain pure crystals of (R)-3-quinuclidinol and recovering the crystals by filtration.

The crystals of (R)-3-quinuclidinol obtained by the present process exhibit more than about 99% purity and enantiomeric excess of greater than about 99.5%.

The process in the present invention increases the loading capacity of the enzymes involved in the conversion of 3-quinuclidinone to (R)-3-quinuclidinol, which results in increase in the yield of the product, increase in production efficiency due to higher production in shorter duration, higher yield and higher purity.

Thus the present inventors have designed a reproducible, economical, industrially feasible and efficient enzymatic process to produce (R)-3-quinuclidinol.

Also the present inventors have successfully reduced reaction time of enzymatic conversion of 3-quinuclidinone to (R)-3-quinuclidinol,

The present invention thus overcomes all the problems and achieves all the object of the invention.

### EXAMPLES

Following examples of the present invention demonstrate the best mode of carrying out the present invention. These examples do not limit the scope of invention in any manner and should be considered as purely illustrative.

### Example 1: Production of (R)-3-quinuclidinol Using Whole Cells

For the bioconversion of 1 g of 3-quinuclidinone, 4 g of cell mass was used in a reaction mix comprising 10 mg of NADP, 6 g of glucose, 10 mg of glucose dehydrogenase in a final volume of 40 ml. Reaction mass was mixed at 150 rpm on a rotary shaker at 25° C.±1° C. for 3-4 h. pH was adjusted intermittently to ˜6.5-7.5 using 20% NaOH solution. The reaction was monitored for the completion by silica gel-Thin Layer Chromatography (TLC).

### Example 2: Production of (R)-3-quinuclidinol Using Cell Lysate

The reaction mixture comprised of 100 ml cell lysate containing at least 4 units of ketoreductase per millilitre and 50 ml cell lysate containing not less than 250 units of glucose dehydrogenase enzyme per millilitre along with 30 mg of NADP and 1.4 g of glucose, 10 g 3-quinuclidinone. The final volume of the reaction mixture was 150 ml. Reaction mass was stirred at 150 rpm on a rotary shaker at 25° C.±1° C. for 3-4 h. pH was constantly adjusted to ˜6.8-7.5—using 20% NaOH. At 4 h, the mixture was sampled to analyze conversion of the substrate 3-quinuclidinone and determine the enantiomeric purity of the product (R)-3-quinuclidinol.

### Example 3: Production of (R)-3-quinuclidinol Using Cell Lysate with Increased Substrate Loading in the Reaction

Reaction mass comprising of ˜60 mL of cell lysate containing at least 4 units of ketoreductase enzyme per milliliter, ˜30 mL cell lysate containing not less than 250 units of glucose dehydrogenase enzyme per millilitre and 30 mg of NADP was used. 3-quinuclidinone was varied from 10.0 g, 12.5 g, 15.0 g, 17.5 g and 20.0 g in a final reaction volume of ˜100 mL. Reaction mass was stirred at 150 rpm on a rotary shaker at 25° C.±1° C. for 3-10 h. pH was constantly adjusted to ˜6.8-7.5 using 20% NaOH. After 10 h the mixture was sampled and analyzed the conversion of 3-quinuclidinone by TLC. Complete conversion was observed in all reactions.

### Example 4: Purification of (R)-3-Quinuclidinol Using Celite

The reaction mixture (100 mL) was alkalified with 20% NaOH solution to pH 12. 5 g celite was added to the alkalified reaction mixture and stirred at 25° C. for 1 h. The reaction mixture was filtered to obtain aqueous solution of product. A 200 mL of n-butanol was added to the aqueous filtrate and mixed vigorously. Aqueous and organic layers were separated and aqueous phase was re-extracted with 200 mL of n-butanol. The n-butanol extracts were pooled, concentrated to dryness and solubilized in toluene at 90° C.-100° C. Hot toluene solution was filtered and the filtrate was allowed to gradually cool to room temperature under constant stirring. (R)-3-quinuclidinol crystals were filtered and dried at 50° C.-60° C. under vacuum. White to off-white crystals were obtained, with more than 99% purity and more than 99.9% e.e.

1-20. (canceled)

21. A process to produce (R)-3-quinuclidinol comprising enzymatic reduction of 3-quinuclidinone or salt thereof using a variant of ketoreductase enzyme derived from Rhodotorula rubra, in the presence of a co-factor regenerating system variant of glucose dehydrogenase derived from Bacillus megaterium; wherein both the variant are in cell lysate; and wherein substrate loading capacity of the variant of ketoreductase enzyme is not less than about 100 g/L.

22. The process as claimed in claim 21, wherein the co-factor is selected from NAD and NADP.

23. The process as claimed in claim 21, wherein the process comprises steps of:
a. reacting 3-quinuclidinone with the cell lysate containing 3-quinuclidinone reductase, in the presence of cell lysate containing glucose dehydrogenase, to give (R)-3-quinuclidinol;
b. extracting and purifying (R)-3-quinuclidinol obtained in step ‘a’.

24. The process as claimed in claim 23, step ‘b’, wherein extracting and purifying (R)-3-quinuclidinol comprising the steps of:
a. basifying/acidifying the reaction mixture, to obtain basified/acidified reaction mixture;
b. adding acetone to the basified/acidified reaction mixture in step ‘a’, filtering the solvent mixture and removing acetone by evaporation to obtain aqueous solution of product;
c. alternatively, adding celite to the basified/acidified reaction mixture in step ‘a’, stirring at 20° C.-30° C. for 20 min to 2 h and filtering to obtain aqueous solution of product;
d. extracting the product from the aqueous solution obtained in step ‘b’ or ‘c’ using n-butanol and concentrating the extract to dryness to obtain the extracted product;
e. solubilizing the extracted product obtained in step ‘d’ in hot toluene at 80° C.-105° C. to obtain solubilized solution; and
f. filtering the solubilized solution obtained in step ‘e’, gradually cooling the filtrate under constant stirring to room temperature to obtain pure crystals of (R)-3-quinuclidinol and recovering the crystals by filtration.

25. The process as claimed in claim 21, wherein substrate loading capacity of the enzyme is not less than 125 g/L.

26. The process as claimed in claim 21, wherein per mL of cell lysate comprises not less than 4 units of ketoreductase enzyme.

27. The process as claimed in claim 21, wherein per mL of cell lysate comprises not less than 250 units of glucose dehydrogenase.

28. A process to enzymatically produce (R)-3-quinuclidinol wherein (R)-3-quinuclidinol produced is not less than 99% pure and has greater than 99.5% enantiomeric excess, wherein the enzyme is in cell lysate; and wherein substrate loading capacity of the enzyme is not less than about 100 g/L.

29. (R)-3-quinuclidinol, produced by the process comprising enzymatic reduction of 3-quinuclidinone or salt thereof using a variant of ketoreductase enzyme derived from Rhodotorula rubra, in the presence of a co-factor regenerating system variant of glucose dehydrogenase derived from Bacillus megaterium wherein both the variant are in cell lysate; wherein substrate loading capacity of the variant of ketoreductase enzyme is not less than about 100 g/L; and the (R)-3-quinuclidinol is not less than 99% pure and having greater than 99.5% enantiomeric excess.

30. The process as claimed in claim 21, wherein substrate loading capacity of the enzyme is not less than 150 g/L.

31. The process as claimed in claim 21, wherein substrate loading capacity of the enzyme is not less than 175 g/L.
